(19) United States
(12) Patent Application Publication
(10) Pub. No.: US 2014/0228455 A1
(43) Pub. Date: Aug. 14, 2014

(54) DETERMINATION OF BACTERIAL INFECTIONS OF THE GENUS RICKETTSIA AND POSSIBLY BORRELIA, IN PATIENTS EXHIBITING SYMPTOMS OF DISEASE AND BEING BLOOD DONORS

(71) Applicants: Kenneth Nilsson, Uppsala (SE); Carl Pahlsson, Uppsala (SE); Jan Carlsson, Uppsala (SE)

(72) Inventors: Kenneth Nilsson, Uppsala (SE); Carl Pahlsson, Uppsala (SE); Jan Carlsson, Uppsala (SE)

(73) Assignee: Alpha Biotech AB, Uppsala (SE)

(21) Appl. No.: 14/343,567

(22) PCT Filed: Sep. 6, 2012
(86) PCT No.: PCT/SE2012/000130

§ 371 Date: Mar. 7, 2014

(30) Foreign Application Priority Data
Sep. 7, 2011 (SE) 1130080-3
Sep. 13, 2011 (SE) 1130082-9

(51) Int. Cl. G01N 33/569

(52) U.S. Cl. CPC G01N 33/56911
USPC 514/789; 435/7.1

(57) ABSTRACT

Disclosed is a method for determining a possible bacterial infection in a patient exhibiting symptoms selected from specific internal symptoms of disease, and/or diffuse and general manifestations of disease, and/or is a blood donor. The method includes: i) determining a) the level of a Rickettsia analyte in a sample 1 from a patient exhibiting symptoms, possibly combined with b) the level of a Borrelia analyte in a sample 2 from the same patient; ii) considering that the symptoms exhibited by the patient involve a) rickettsiosis if the level of the Rickettsia analyte in sample 1 is elevated, and b) borrelios if the level of the Borrelia analyte in sample 2 is elevated. For blood donors step, (ii) means that whole blood and blood fractions are considered to be infected with Rickettsia and/or Borrelia, respectively, when these levels are elevated. Also disclosed is a corresponding kit.

### TECHNICAL FIELD

The invention relates to a method and a kit for the determination of bacterial infection of a patient a) having symptoms that potentially may be associated with infection by bacteria of the genus Rickettsia and/or the genus Borrelia, or b) being a blood donor. The invention also relates to the use of the method and the kit in the treatment of infection by one or more of these bacteria. Bacterial infections in particular refer to on-going infections

The patients are primarily vertebrates such as mammals with primary emphasis of humans and domestic vertebrates including domestic mammals such as horses. In the context of the invention blood donors are considered to be patients and comprises individuals donating whole blood and/or only certain blood fractions, e.g. plasma.

### TECHNICAL BACKGROUND

The typical route for infection by Rickettsia and/or Borrelia comprises that an individual is attacked by a native living vector carrying the bacteria which subsequently is transferred to the individual. The attack typically involves a bite of the vector followed by transfer of the bacteria via the bite. Potentially other routes may be possible, such as via inhalation of the vector, Rickettsia and/or Borrelia bacteria, faeces of the vector etc, blood transfusions etc. Typical vectors are athropods and are in particular found amongst a) the arachnids, such as an arcari, e.g. a tick or a mite, or b) insects such as a siphonaphtera (fleas) or a phthiraptera (louse). The typical vectors for Rickettsia are ticks, mites, fleas, and louse. The typical vectors for Borrelia are ticks and fleas. For both of the bacteria the vector utilized vary with species of bacteria. In the literature it is mentioned that Rickettsia may also be associated with leeches and protists as vectors.

Rickettsia infection (rickettsiosis) at an early stage is typically manifested as a more or less pronounced skin rash. For Rickettsia of the spotted fever group (SFGR) the rash is typically spotted and combined with fever. There may also be diffuse and general symptoms of disease such as fatigue, myalgia/arthralgia, headache, neck pain, numbness, vertigo, concentration difficulties, nausea, radiating pain, fever, loss of appetite, weight loss etc. The symptoms often vary between bacterial species which usually are spread by different vector species, individuals infected etc. Immediate and safe diagnosis is difficult. For certain Rickettsia species the spotted rash is less pronounced, less frequent and/or absent (=possible SFGR species)

SFGR species which often results in a pronounced spotted rash are for instance R. rickettsii and R. japonica which are associated with Rocky Mountain spotted fever and Japanese spotted fever, respectively (spotted Rickettsia=spotted fever SFGR). Patients infected with R. helvetica or other possible spotted fever Rickettsia typically present with vague and diffuse symptoms (Fournier et al., 2004) (non-spotted Rickettsia=non-spotted SFGR). An inoculation eschar and erythematous rash are occasionally seen (Parola et al., 2001; Fournier et al., 2004; Nilsson, 2009). R. helvetica is the spotted fever Rickettsia which is the most frequent species found in European ticks.

The fact that an SFGR infection is considered relatively uncommon in many geographical areas (e.g. Europe) and initially may be manifested with vague and general symptoms has meant that the possibility for Rickettsia infection easily is overlooked. If samples have been assayed for bacterial infections, the testing has been for a standard panel of infectious agents other than Rickettsia, i.e. other bacteria (e.g. Borrelia), viruses, fungi etc. This is illustrated in a number of scientific articles describing cases in which various treatments have failed until one in a late stage has realized the involvement of a Rickettsia infection, or saved samples have been assayed for Rickettsia after recovery or death of the patient.

Other main groups of Rickettsia are the typhus group Rickettsia and the scrub typhus group.

A Borrelia infection (Borrelios, Lyme disease, Lyme Borrelios) is typically considered when patients exhibit rashes in the form of erythema migrans (a growing circular rash around the bite of a vector carrying the bacteria) and/or diffuse and general symptoms of the same or similar kind as discussed above for a Rickettsia infection. The Borrelia species involved are called Borrelia sensu lati. Another kind of Borrelia is B recurrentis which upon infection gives recurrent fever which is another kind of borrelios. In contrast to ricketsiosis, testing for Borrelia has become more or less a standard for patients with these symptoms. This means that on-going rickettsiosis normally not is detected leading to unnecessary suffering and the risk for development of serious sequelae for this group of patients.

The anti-microbial agents that are effective for treating Borrelia infections are typically of the β-lactam type and include selected penicillins, and are different from those that are effective for treating Rickettsia infection (tetracycline-type). To avoid unnecessary suffering for the patient and improve the cost-effectiveness of a treatment, it is import to use safe methods for early determination of the etiologic agent and for selecting the correct treatment regimen.

There are indications in the literature that Borrelia and/or Rickettsia bacteria may be involved in the development of autoimmune, neurodegenerative and psychotic conditions and/or diseases, potentially Alzheimer's disease, Parkinson's disease, Huntington's disease, Amyothropic lateral sclerosis (ALS), Multiple sclerosis (MS), etc. See for instance Bruce Fife, N.D., 2011. This suggests that it may be worthwhile to test also these patients, in particular at an early stage of their disease, for ricketsiosis and/or borrelios. In the context of the invention, autoimmune, neurodegenerative and psychotic conditions and diseases are considered to be symptoms of category (A) below.

### OBJECTS OF THE INVENTION

The main object of the invention comprises to overcome the problems discussed above and to provide improved methods for determination of a possible infection by Rickettsia and/or Borrelia bacteria in a patient which
a) exhibits symptoms selected amongst:
   A) specific internal symptoms of disease, such as organ specific symptoms, nerve inflammation (neuritis), autoimmune diseases or conditions, neurodegenerative diseases or conditions, psychotic diseases or conditions, and/or
   B) visible skin manifestations which are associated with infection by a bacterium of the genus Rickettsia and/or the genus Borrelia, and/or
   C) diffuse and general manifestations of disease, and/or
   D) manifestations indicating that the patient have been attacked by a living native vector that can be used for infection by a bacterium of the genus Rickettsia and/or the genus Borrelia, and/or
b) is a blood donor.

These objects in particular apply to the determination of infection by Rickettsia of the spotted fever group (SFGR) and possibly also to the determination of infection by Borrelia. SFGR includes either one or both of spotted fever SFGR and non-spotted SFGR. Blood donors normally do not exhibit any of the symptoms (A)-(D).

The symptoms of group (A) may or may not involve vasculitis and/or neuritis. Organ specific symptoms means limited to one or more internal organs, e.g. nerves, heart, liver, kidneys, brain, lungs, local muscles, local or certain type of blood vessels, intestines, colon etc. The symptoms typically indicates inflammation, e.g. meningitis, perimyocarditis in particular chronic forms thereof, facial paresis, e.g. Bell's paresis, hepatitis, nevritis causing symptoms of loss of function of for example nerves, such as cranial nerves, or pain from different and/or specific parts of the body etc. The term “internal” means within the body of a patient and excludes skin manifestations of the kind defined for group (B).

Visible skin manifestations of group (B) may or may not be recognized to be associated with infection by bacteria of the genus Rickettsia and/or the genus Borrelia. The expression primarily refers to rashes and/or eschars associated with rickettsiosis and borrelios, i.e. spotted rash, eschars and erythema migrans.

Group (C) include symptoms such as fatigue, myalgia/arthralgia, headache, neck pain, numbness, vertigo, concentration difficulties, nausea, radiating pain, fever, loss of appetite, weight loss etc. In other words symptoms that may be associated with a number of disease states other than rickettsiosis and borrelios.

Manifestation of group (D) includes any sign of a vector attack on the patient It may be the sign of a bite with or without signs if inflammation around the site of the bite. It may be alteration in the lungs in case the attack is via the lungs.

A second main object is to provide improvements in the therapy of the abovementioned symptoms and diseases and includes improvements in the treatment of rickettsiosis and/or borrelios.

Subobjects are to provide suitable methods and reagent kits for assaying Rickettsia and/or Borrelia analytes in biological samples deriving from patients that exhibit the symptoms, manifestations and diseases mentioned above. Subobjects also include providing increased patient security in administration of blood and blood products such as different fractions of whole blood.

### THE INVENTION

The present invention is based on the inventors finding that rickettsiosis in several cases is capable of causing meningitis and chronic perimyocarditis and other internal symptoms of vasculitis or nervous system involvement, e.g. nerve inflammation that may cause symptoms of loss of function of for example cranial nerves or pain from different parts of the body or hepatitis. This suggests that diagnostics for rickettsiosis of a patient with diffuse and rather general clinical symptoms or internal focal manifestations from different organs should allow for early detection and prevention of later and more serious sequelae, e.g. by early therapy with an anti-microbial agent effective against ricketsiosis.

The results presented in the experimental part illustrate that it is not uncommon that patients diagnosed for Borrelia infection may in fact also suffer from infection by other etiological agents. This finding is likely to explain the fact that it is not uncommon that these patients respond poorly to treatment with anti-microbial agents for Borrelia.

### Method of Determination (1^{ST} Aspect)

This aspect of the invention is a method for determining a possible infection by Rickettsia and/or Borrelia bacteria in a patient which
a) exhibits symptoms selected amongst:
   A) specific internal symptoms of disease, such as organ specific symptoms, nerve inflammation (neuritis), autoimmune diseases or conditions, neurodegenerative diseases or conditions, psychotic diseases or conditions, and/or
   B) visible skin manifestations which are associated with infection by a bacterium of the genus Rickettsia and/or the genus Borrelia, and/or
   C) diffuse and general manifestations of disease, and/or
   D) manifestations indicating that the patient have been attacked by a native vector that can be used for infection by a bacterium of the genus Rickettsia and/or the genus Borrelia, and/or
b) is a blood donor.

The symptoms, manifestations and diseases mentioned in (A)-(D) have been further illustrated under “Objects of the Invention” and “Technical Background”.

A characteristic feature of an embodiment (1^{st}) of the invention comprises the steps of:
i) determining
   a) the level of a Rickettsia analyte in a biological sample 1 which derives from a patient exhibiting symptoms according to (A) and/or being a blood donor, possibly combined with
   b) the level of a Borrelia analyte in a biological sample 2 which derives from the same patient,
ii) considering that the symptoms exhibited by said patient involve
   a) rickettsiosis if the level of the Rickettsia analyte in sample 1 is elevated compared to corresponding samples from healthy individuals, and
   b) borrelios if the level of the Borrelia analyte in sample 2 is elevated compared to corresponding samples from healthy individuals.

This first embodiment of the inventive method thus comprises that a patient which exhibits symptoms according to (A) is always tested for Rickettsia infection by assaying for Rickettsia analyte in sample 1, optionally in combination with testing for Borrelia infection by assaying for Borrelia analyte in sample 2. The patient may in this variant of the invention exhibit one or more of the symptoms (C)-(D) in addition to (A).

Another embodiment (2^{nd}) comprises the steps of:
i) determining
   a) the level of a Rickettsia analyte in a biological sample 1 which derives from a patient exhibiting symptoms according to at least one of the symptoms (A)-(D) and/or being a blood donor, combined with
   b) the level of a Borrelia analyte in a biological sample 2 which derives from the same patient,
ii) considering that the symptoms exhibited by said patient involve
   a) rickettsiosis if the level of the Rickettsia analyte in sample 1 is elevated compared to corresponding samples from healthy individuals, and
   b) borrelios if the level of the Borrelia analyte in sample 2 is elevated compared to corresponding samples from healthy individuals.

Normal rules for serologically determination if an infection is ongoing apply. In other words repeated testing is needed on samples taken from the same patient at two different times with a time lapse of about 3-10 weeks between the samples. The criteria for on-going infection is that the titers of the two samples should differ with factor ≧2, such as ≧3 or ≧4 (increase from first to second sample) and with a factor of ≦½, such as ≦⅓ or ≦¼ (decrease in titer from first to second sample) and/or with at least one of the samples having a titer of at most 1:128, with preference for at most 1:256 (dilution factor for diluting the original sample to the cut off limit for the assay used).

This second embodiment thus comprises that the patient which exhibit at least one of the symptoms (A)-(D) is tested for both Rickettsia infection and Borrelia infection, i.e. sample 1 and sample 2 are imperatively assayed for Rickettsia analyte and Borrelia analyte, respectively, if at least one of the symptoms (A)-(D) are at hand for the patient.

For blood donors step (ii) means that whole blood and blood fractions obtained from a blood donor are considered to be infected with Rickettsia and Borrelia, when these levels are elevated in samples 1 and 2, respectively. This applies to both the 1^{st} and the 2^{nd} embodiment.

The comparison in steps (ii.a) and (ii.b) means that the level of analyte in samples from the patient is compared with the level of analyte in samples from healthy individuals, i.e. with normal levels determined at the same occasion as or at an occasion different from the determination of the level(s) in patient samples.

The original samples from the patient should have been taken while the patient's immune response to Rickettsia and/or Borrelia infection is on-going, and in particular before the symptoms of (A)-(D) have disappeared, e.g. by death or recovery of the patient. These time frames also apply to the time at which steps (i)-(ii) (=assaying steps for Rickettsia and/or Borrelia analytes) are carried out. Steps (i)-(ii) are preferably also carried out before any anti-microbial treatment is initiated or, if such a treatment has been initiated, subsequent to or during the treatment as a follow up of the treatment in order to check for the appropriateness of the treatment or of the antimicrobial used. In other words the inventive method encompasses in one variant that steps (i) and (ii) are carried out on samples taken at two different times from the same patient preferably with a time lapse of about 3-10 weeks between the samples/times. This includes a first time in an initial phase of the illness, e.g. at the initial contacts with the health care system and/or before antimicrobial treatment has been initiated, and a second time on a sample taken 3-10 weeks after the first sample. The second testing is for the same analyte(s) as the first testing with preference for testing for both the Borrelia analyte and the Rickettsia analyte the second time. This in particular enables the determination of on-going infection. See elsewhere in this specification.

Blood donors may be tested for either one or both of rickettsiosis or borrelios. The original samples to be tested are typically obtained directly from the donor or from blood or blood fractions donated by the donor.

A Rickettsia analyte is specific for Rickettsia infection and appears in a patient infected with Rickettsia bacteria. This analyte is also called Rickettsia specific analyte.

A Borrelia analyte is specific for Borrelia infection and appears in a patient infected with Borrelia bacteria. This analyte is also called Borrelia specific analyte.

### Assay Formats for the Determination in Step (i)

Step (i) typically comprises the imperative determination of the Rickettsia analyte (step i.a) by one biospecific assay and the optional determination of the Borrelia analyte (step i.b) by another biospecific affinity assays, each of which comprises the substeps of
(i.a/b.A) formation of an affinity complex containing the analyte and an affinity counterpart to the analyte (anti-analyte) by reacting the analyte with the anti-analyte, and
(i.a/b.B) measuring during or subsequent to substep (i.A) the amount of the complex formed,
i.a/b.C) taking the formation of said complex as an indication of an elevated level of Rickettsia analyte and Borrelia analyte, respectively, in the original biological sample.
   The measurement in substeps (i.a.B) and (i.b.B) may comprise measurement of: I) the affinity complex formed, and/or II) the analyte not incorporated in the complex, and/or III) the anti-analyte not incorporated in the complex. The term “amount” in this context comprises that one measures the presence and/or the amount of the complex.

The conditions for the complex formation are selected such that the amount of complex formed and/or the amount of anti-analyte not incorporated in the complex and/or the amount of analyte not incorporated in the complex will be a function of the amount of analyte in the original sample. This is according to well-established affinity assay formats which includes that a predetermined amount of anti-analyte is allowed to react with the analyte for formation of the complex.

Useful assay formats may be classified according to:
A) the type of analyte ((i)-(ii) below meaning that the assay is an immunoassay including as subgroups a) an antigen immunoassay in which the analyte is a Rickettsia analyte or a Borrelia analyte and b) an antibody immunoassay in which the analyte is an anti-Rickettsia antibody or an anti-Borrelia antibody,
B) utilizing a competitive or a non-competitive biospecific affinity assays,
C) utilizing an affinity reactant exhibiting a reporter group, typically an analytically measurable group (=label),
D) utilizing an affinity reactant which a) is immobilized or immobilizable to a solid phase, or b) is dissolved in the in the reaction mixture at the start of the assay,
E) utilizing flow conditions or non-flow conditions
   A. The analyte

The Rickettsia analyte derives from one or more Rickettsia species which typically is pathogenic, e.g.
a) spotted fever group Rickettsia (SFGR), such as Rickettsia rickettsii, R. akari, R. conorii, R. sibirica, R. sibirica mongolotimonae, R. australis, R. felis, R. japonica, R. africae, R. hoogstraalii, R. parkeri, R. species 364D, R. aeschlimannii, R. heilongjiangenesis, R. helvetica, R. honei, R. marmioni, R. massilia, R slovaca etc,
b) typhus group Rickettsia, such as R. prowazekii, R. typhi etc,
c) scrub typhus group Rickettsia, such as R. tsutsugamushi (name changed to Orientia tsutsugamushi) etc, etc

An SFGR analyte may derive from spotted fever SFGR or non-spotted fever SFGR. The typical vector for Rickettsia infection is an arthropod, e.g. a tick, a mite, a flea a louse, and may also be associated with leeches and protists.

The Rickettsia analyte (derives from the patient) is selected amongst i) Rickettsia antigen and/or ii) anti-Rickettsia antibody. Present preference is to measure analytes according to (ii).

The Borrelia analyte typically derives from one or more pathogenic Borrelia species, e.g. Borrelia burgdorferi sensu lati, i.e. B. burgdorferi, B. afzeli, B. garini, B spielmaii etc, B recurrentis, etc. The typically vectors for Borrelia sensu lati are ticks and for B recurrentis are fleas.

The Borrelia analyte may be selected amongst i) Borrelia antigen and ii) anti-Borrelia antibody etc. Present preference is to measure analytes according to (iii)

Preferably the Borrelia analyte and the Rickettsia analyte are of the same kind, i.e. both of them are antigens (ii), or antibodies (iii), with present preference antibodies (class (iii).

Rickettsia and Borrelia bacteria as such may also be used as analytes because they contain analytes of class (i) and (ii). Without being lysed, the whole bacteria may be measured as an antigen analyte in the two biospecific affinity assays used in the invention because the surface structure of Rickettsia and Borrelia expose a) lipopolysaccharides and lipopolysaccharide-like compounds that are genus specific for Rickettsia and Borrelia, respectively, and b) proteins that are genus, group or species specific for these bacteria. Also other genus, group or species specific components of these bacteria may be measured as analytes in the invention.

In the preferred embodiment at the priority date one or most preferably both of the Rickettsia analyte and the Borrelia analyte are antibodies, i.e. anti-Rickettsia antibody analyte and anti-Borrelia antibody analyte, respectively. It is most appropriate that measurements of these antibody analytes include measuring antibodies of the IgG and/or IgM class.

Antigen analytes (ii) and antibody analytes (iii) are typically measured by immunoassays. This means that the anti-analytes used as affinity reactants for measuring
a) antigen analytes are anti-Rickettsia antibody and anti-Borrelia antibody, respectively, and
b) antibody analytes are Rickettsia antigen and Borrelia antigen, respectively.

The term analyte include analyte-derived entities obtained by processing an original sample prior to the actual quantification step (step (i)) as discussed above. An analyte-derived entity formed during this kind of processing is typically formed in a level and amount that is a function of the level and amount of the starting analyte in the starting/original sample. If not otherwise indicated the term analyte include analyte-derived entities in this specification.

### B) Competitive or a Non-Competitive Biospecific Affinity Assays.

Competitive assays mean that an analyte and an analyte analogue are allowed to compete with each other about the same binding sites of an anti-analyte. These binding sites and typically also the anti-analyte are typically present in a limiting molar amount. The analyte analogue typically exhibits a reporter group that as such may be measurable (=a label) or render the formed complex immobilized or immobilizable to a solid phase. The amount of complex formed containing the analyte analogue and/or the amount of analyte analogue not incorporated in the complex will be a measure of the amount of analyte in the sample. A competitive assay may be simultaneous or sequential where simultaneous means that the analyte and analyte analogue are reacting simultaneously with the anti-analyte and sequential means that the analyte and the analyte analogue are reacted one after the other with the anti-analyte. A sequential competitive assay may also be arranged as a displacement assay.

Non-competitive assays are preferred in the invention and encompass biospecific assays that are not competitive. They preferably comprise that the analyte is allowed to react with an unlimiting amount of anti-analyte for formation of the affinity complex. The anti-analyte typically exhibits a reporter group selected from the same kind of groups as may be present on an analyte-analogue used for a competitive assay.

The preferred non-competitive formats are sandwich formats and comprise formation of an at least ternary affinity complexes with the analyte layered between two anti-analytes having the same or different binding specificity for the analyte. Each of the two anti-analytes typically exhibits a reporter group that may differ for the two anti-analyte reactants. The reporter group on one of the anti-analytes may for instance render the anti-analyte immobilized or immobilizable, while the reporter group on the other anti-analyte may be a label rendering this anti-analyte analytically measurable for facilitating measurement of the formed complex.

In the invention it is preferred that the Rickettsia assay and the Borrelia assay have the same format with respect to being competitive or non-competitive with highest preference for non-competitive, such as sandwich formats.

### C. Bioaffinity Assays Utilizing Labeled Affinity Reactants.

The preferred bioaffinity assay formats utilize labeled affinity reactants for measuring the formed complex. The label is typically a reporter group which is enzymatic, radioactive, fluorescent/fluorogenic, luminescent, exhibits bioaffinity etc. This kind of reporter groups also includes colloidal particles, e.g. gold particles, carbon particles etc. The label may be present on an anti-analyte, an analyte-analogue or some other affinity reactants which may be used for measuring the affinity complex containing the analyte and the anti-analyte.

The label used in the Rickettsia assay may be different from the label used in the Borrelia assay, or be the same. It is preferred that the label used in each of the Rickettsia assay and the Borrelia assay can be measured by the same instrument in which case the two labels used may be different or identical.

Bioaffinity assay formats which do not utilize labeled affinity reactants may also be used.

D) Affinity Reactants which are Immobilized or Immobilizable to a Solid Phase

Suitable affinity assay formats may utilize affinity reactants that are immobilized or immobilizable to a solid phase. This includes that the reactant is part of a solid phase or is capable of being transformed to a solid phase (=insolubilized or insolubilizable).

Suitable solid phase may be in the form of porous or non-porous particles or a porous monolithic bed. A porous monolithic bed may be in the form of a plug, a column, a strip, a capillary, a tube etc, for instance. Solid phases in the form of particles may be used packed to a porous bed or suspended in an aqueous liquid. Solid phases may contain an immobilized affinity reactant, such as an immobilized anti-analyte or an immobilized analyte analogue.

Provided each of the Rickettsia assay and the Borrelia assay utilizes a solid phase, it is preferred that the same kind of solid phase is used in both assays, i.e. the same kind of suspended particles or the same kind of porous bed, the same kind of immobilized affinity reactant, e.g. an immobilized analyte analogue or an immobilized anti-analyte in each of the two assays, etc.

### E) Flow Conditions or Non-Flow Conditions (=Static Conditions)

In affinity assay formats which are suitable for the invention, the reaction between the analyte and the anti-analyte may take place under flow conditions or under non-flow conditions (often called static conditions).

Formats comprising flow conditions typically utilize a device comprising a flow-through matrix in which there is defined a flow line for transporting a liquid flow containing the analyte. The flow line typically contains a capturing zone containing a solid phase to which an anti-analyte is pre-immobilized, i.e. immobilized prior to passage of the analyte. An immobilized affinity complex containing the analyte and the anti-analyte will be formed when the liquid flow containing the analyte passes through the zone. This complex is measured and the amount of analyte in the starting sample calculated.

The solid phase in the capturing zone is typically in the form of a porous bed as defined above. The flow through matrix upstream and/or downstream of the capturing zone may be of the same material as the solid phase in the capturing zone, or of a different material.

The flow line may be defined as a covered or uncovered channel in a planar substrate, as a planar strip of a porous sheet material allowing transportation of the liquid flow etc.

Suitable assay formats which are utilizing non-flow conditions are typically run in a reaction vessel, for instance microtitre wells, cultivation vessel etc and with or without utilization of a solid phase.

The inventors consider that each of the Rickettsia assay and the Borrelia assay preferably will utilize reaction under flow conditions for substeps (i.a.A and i.b.A) and comprise the substeps of
A) providing a device having a flow line with a capturing zone containing a porous solid phase exposing an immobilized analyte analogue or more preferably an immobilized anti-analyte, and
B) passing the analyte through the zone under competitive conditions or more preferably under non-competitive conditions for the formation of an immobilized affinity complex containing the anti-analyte and the analyte.

The device may contain a separate flow line for each assay. More preferably there is a common flow line for the two assays, typically with two separate capturing zones or a common capturing zone. A common flow line with two capturing zones is illustrated in the experimental part.

In conclusion the preferred variants of the invention comprises that the biospecific affinity assays for the Rickettsia analyte and the Borrelia analyte, respectively, are the same with respect to at least one key features (A′)-(E′) below. The highest preference is that all of these features are the same with respect to:
A′) the type of analyte (i)-(iii),
B′) utilizing a non-competitive biospecific affinity assays, such as a sandwich biospecific affinity assays,
C′) utilizing an affinity reactant which is labeled to determine the formation of said affinity complex, with preference for the labeled affinity reactant being a labeled anti-analyte,
D′) utilizing an affinity reactant which is immobilized or immobilizable to a solid phase, with preference for the immobilized or immobilizable reactant being an anti-analyte,
E′) utilizing flow conditions for the formation the affinity complex with a liquid flow containing the analyte passing through a capturing zone containing the anti-analyte in immobilized form.

It is further preferred to form the affinity complex containing the Borrelia analyte and the affinity complex containing the Rickettsia analyte in the same starting reaction mixture (=multiplexed assay) and/or essentially simultaneously or essentially consecutively.

### The Biological Samples Used in Step (i)

The biological fluid samples 1 and 2 used in step (i) are of a kind in which the analyte is known to be present when an individual is infected by Rickettsia and/or Borrelia, respectively.

The biological fluid sample may be a vertebrate fluid sample, such as a mammalian fluid sample or a domestic mammalian fluid sample, e.g. a horse fluid sample.

The biological samples 1 and 2 of step (i) may be original samples from the patient to be tested. Step (i) preferably include preprocessing of the sample to a suitable form before the actual formation of the complex to be measured is taking place. The original sample may thus be a tissue sample or a sample of a body fluid, e.g. blood (including plasma or serum), urine, cerebrospinal fluid (=CSF), etc including preprocessed such samples. Blood samples, such as whole blood, serum and plasma samples, and CSF samples typically are likely for analytes that are antigen, or antibodies (class (i)-(ii) above). Urine samples are useful for antigen analytes (class (i) above) which are sufficiently small for passing into urine by renal filtration.

Preprocessing of a sample includes
a) fractionation of an original sample to remove disturbing components e.g. by desorption of disturbing components,
b) increasing the amount and/or concentration of the analyte,
c) formation of an analyte-derived entity facilitating measurement according to a desired assay format,
d) increasing availability of an analyte for reacting with the anti-analyte used in an assay
e) etc.

Preprocessing according to (a) includes fractionation of whole blood into plasma or serum containing the analyte. Preprocessing according to (b) includes conventional concentrating for increasing the amount of the analyte e.g. by adsorption/desorption of an analyte, dialysis, culturing of samples containing viable analyte bacteria etc. Preprocessing according to (c) includes formation of a suitable affinity complex that is analytically detectable/measurable in a desired assay format. Processing according (d) includes lysis of intact analyte bacteria, for instance.

The Rickettsia analyte and the Borrelia analyte are normally present in the same kind of sample when an individual is infected by both Rickettsiaa and Borelia. This means that the determination of each of the two analytes is preferably carried out on the same kind of sample. Sample 1 then preferably is equal to sample 2 and the formation of the affinity complex discussed for the assay formats discussed below can be carried out simultaneously or consecutively (in sequence) in a common reaction mixture or in two separate reaction mixtures (one for the Rickettsia analyte and one for the Borrelia analyte) containing different aliquots from the same original sample or from two different original samples.

The sample to be assayed according to the invention and possibly containing the Rickettsia and/or Borrelia analyte may also derive from blood/plasma transfusion vessels/bottles/bags, i.e. from blood/plasma donors. In this case none of the symptoms (A)-(D) are normally observed in the patient (=blood/plasma donor).

### Kit of Reagents (2^{ND} Aspect)

This aspect is a kit comprising A) a Rickettsia reactant specific for detecting a Rickettsia analyte and B) a Borrelia reactant specific for detecting a Borrelia analyte.

The Rickettsia specific reactant may be selected amongst affinity counterparts to the Rickettsia analytes discussed above, i.e. this reactant is an anti-Rickettsia analyte. This reactant may thus be
i) an anti-Rickettsia antibody which is specific for a Rickettsia specific antigen (specific for an antigen analyte), or
ii) a Rickettsia specific antigen which is an affinity counterpart to an anti-Rickettsia antibody formed in an individual as a consequence of a Rickettsia infection (specific for an antibody analyte).

The Rickettsia specific antigen referred to above is typically part of the surface structure of Rickettsia, for instance a structure exposed to the immune system of an infected individual and typically comprises a lipopolysaccharide (LPS) and/or a protein. The reactant thus should be genus, group or species specific for Rickettsia when used in the invention.

The Borrelia specific reactant may be selected amongst affinity counterparts to the Borrelia analytes discussed above, i.e. this reactant is an anti-Borellia analyte. It may thus be
i) an anti-Borrelia antibody which is specific for a Borrelia specific antigen (specific for an antigen analyte), and
ii) a Borrelia specific antigen which is an affinity counterpart to an anti-Borrelia antibody formed in an individual as a consequence of a Borrelia infection (specific for an antibody analyte).

The Borrelia specific antigen referred to above is typically part of the surface structure of Borrelia, for instance a structure exposed to the immune system of an infected individual and typically comprises a lipopolysaccharide-like compound (LPS-like compoumd) and/or a protein. The reactant thus should be genus, group or species specific for Borrelia when used in the invention.

Each of the Rickettsia and the Borrelia specific reactant may comprise a mixture of different reactants each of which may have its own group or species specificity with respect to affinity binding to Rickettsia and Borrelia, respectively. If immobilized to a solid phase they may be physically separated from each other.

The kit of the invention thus comprises one analyte specific reactant of each of the two assays. This reactant is either an anti-analyte or an analyte analogue. In preferred variants there is also included a second analyte specific reactant for each assay. For non-competitive sandwich assays this second reactant is preferably an anti-analyte and for competitive assays it is an analyte analogue. In other words preferred variants of the kit of the invention comprise one or two Rickettsia specific reactants and one or two Borrelia specific reactants. The number of analyte specific reactants is preferable the same for the two assays.

In preferred variants of the kit the corresponding Rickettsia specific and Borrelia specific reactants of the kit belongs to the same class (i) or (ii) with further preference for a Rickettsia antigen and a Borrelia antigen. For non-competitive assay formats such as sandwich assays these antigen reactants may be combined with an anti-(anti-Rickettsia antibody) and anti-(anti Borrelia antibody), respectively, as additional affinity reactants. These reactants may be immobilized, immobilizable and/or labeled as discussed above.

The kit may also comprise a device for carrying out the two assays of the invention. This device preferably comprises a suitable reaction vessel which may be part of a flow line in case affinity reactions are to be carried out under flow conditions. As also discussed elsewhere in this specification a device adapted for flow conditions thus typically comprises
a) a flow line which is common for the two assays with a capturing zone which is common for the two analytes or with two separate capturing zones (one for each analyte), or
b) two separate flow lines each of which has a capturing zone.

In addition to be used in the first aspect of the invention, the kit may also be used for testing blood/plasma in transfusion vessels, bags, bottles etc.

### The Use of the Method in the Treatment of Rickettsia and/or Borrelia Infection (3^{RD} Aspect)

This aspect is a method for the treatment of a patient exhibiting at least one of the symptoms (A)-(D) given above. The method of the third aspect comprises the steps of:
(i) carrying out the method of the first aspect of the invention on the patient as described above, and subsequently
(ii) treating the patient with an anti-mirobial agent effective against the bacteria determined in step (i).

Suitable anti-microbial agents are discussed elsewhere in this specification. Subaspects of this main aspect are as defined for the first aspect. For carrying out step (i) a kit according to the second aspect of the invention is preferably used. This method in particular apply to the determination and treatment of on-going infection

This aspect also comprises the method and kit of the 1^{st} and 2^{nd} aspects, respectively, for use in the treatment of Rickettsia and/or Borrelia infection.

### EXPERIMENTAL PART

### Clinical Study 1

This is complentary study based on a sero survey study of 220 patients, with proven tick bite and signs of infection of “Borreliosis” (erythema chronica migrants, ECM) performed by Eliasson et al. We have afterwards retested sera from patients of this study for serological response for Rickettsia 1 infection. It was then shown that 20 of the patients show sero conversion for Rickettsia 1 infection. This means that in 10% of the cases infection by Rickettsia may be involved. This is a completely different organism than Borrelia and not sensitive for the normal antibiotics that are used to treat Borrelia infections. Samples from Rickettsia-positive patients taken a number of weeks after their initial contact with their doctors were tested and compared with samples taken at their initial contact with the doctor. For each patient the results showed a significant change in titer between the two samples which strongly suggests an on-going infection. See ref 6 (Lindblom et al.)

### Clinical Study 2

At a clinical microbiological laboratory that covers a country side area in Sweden (Falu hospital) 84 patients with “Bells paresis” are analysed for Borrelia infection during one year. One sequel of Borrelia infection are “Bells paresis” or facialis paresis. The results from this study are that one patient have antibodies against Borrelia burgdorferi sensu late, however, 14 of the patients were positive for Rickettsia infection.

### Lateral-Flow Assay

A lateral flow assay is believed to be preferred at the filing of the present application. Preferred embodiments of these kinds of assays will now be described.

A lateral-flow assay consists of a detection strip made of nitrocellulose that is flanked at one end by a application zone were carbon black or colloidal gold-labeled anti-human antibody (reagent fluid) are mixed with the patient sample. Antigen is prepared from an isolate of a Rickettsia helvetica and a Borre iagnostiorferi bacterial culture. The suspension is centrifuged to remove cell debris, and the supernatant containing the antigen is filtered. The Rickettsia antigen is deposited onto the nitrocellulose strip as a 1-mm narrow line 10 mm upstream of the application zone, 5 mm further upstream a new line with Borrelia antigen is deposited. Human antibodies are deposited in a third line parallel to and upstream of the antigen lines to function as a reagent control. The composite is backed by a support and cut into 5-mm-wide test strips to fit a plastic housing with a round sample application well positioned above the application zone and a square detection window positioned above the detection strips. The amounts of antigen and detection reagent are optimized in a step-by-step procedure with a panel of positive and negative control sera. The assay is performed by the addition of 5 μl of undiluted serum followed by the addition of 130 μl of reagent fluid. The sample fluid can consists of phosphate-buffered saline containing 0.66 mg of bovine serum albumin per ml and 3% Tween 20. The assay is scored positive when a distinct staining of the antigen line/or lines is observed. When no staining is observed the test is negative.

While the invention has been described and pointed out with reference to operative embodiments thereof, it will be understood by those skilled in the art that various changes, modifications, substitutions and omissions can be made without departing from the spirit of the invention. It is intended therefore that the invention embraces those equivalents within the scope of the claims which follow.

### REFERENCE LIST

A. Articles with One or More of the Inventors as Authors
1. Elfving K. et al., Dissemination of spotted fever rickettsia agents in Europe by migrating birds. PloS One. 5(1) (2010 Jan. 5) e8572
2. Elfving K. et al., Seroprevalence of Rickettsia spp. Infection among tick-bitten patients and blood donors in Sweden. Scand. J. Infect. Dis. 40(1) (2008) 74-7
3. Hajem N. et al., A study of the antigenicity of Rickettsia helvetica proteins using two-dimensional gel electrophoresis. APMIS 117(4) (2009 April) 253-62
4. Nilsson K. et al., Rickettsia helvetica in patient with meningitis, Sweden, 2006. Emerg. Infect. Dis. 16(3) (2010 March) 490-2
5. Lindblom A. et al., Rickettsia felis infection in Sweden: report of two cases with subacute meningitis and review of the literature. Scand. J. Infect. Dis. 42(11-12) (2010) 906-9
6. Lindblom A et al., Seroprevalence of spotted fever and coinfections with other tick-borne agents among habitants in central and southern Sweden. Eur. J. Clin. Microbiol. Infect. Disease (in press 2012)
7. Nilsson K., Septicaemia with Rickettsia helvetica in a patient with acute febrile illness, rash and myasthenia. J. Infect. 2009 January; 58(1) (2009 January) 79-82
8. Nilsson K. et al., Evidence of Rickettsia spp. Infection in Sweden: a clinical, ultrastructural and serological study. APMIS 113(2)(2005 February) 126-34
9. Nilsson K. et al., Demonstration of intracellular microorganisms (Rickettsia spp., Chlamydia pneumoniae, Bartonella spp.) in pathological human aortic valves by PCR. J. Infect. 50(1) (2005 January) 46-52
10. Nilsson K, et al., Presence of Rickettsia helvetica in granulomatous tissue from patients with sarcoidosis. J. Infect. Dis. 15;185(8) (2002 April) 1128-38
11. Nilsson K, et al., Association of Rickettsia helvetica with chronic perimyocarditis in sudden cardiac death. Lancet. 354(9185) (1999 October) 1169-73
12. Nilsson K, et al., Rickettsia helvetica in Ixodes ricinus ticks in Sweden. J. Clin. Microbiol. 37(2) (1999 February) 400-3
13. Nilsson K, et al., Characterization of a spotted fever group Rickettsia from Ixodes ricinus ticks in Sweden. J. Clin. Microbiol. 35(1) (1997 January) 243-7
14. Påhlsson C., Novel peptide diagnostic reagent and kit for the detection of rickettsiosis. WO 9942479 (publ 1999)
15. Severinsson K et al., Detection and prevalence of Anaplasma phagocytophilum and Rickettsia helvetica in Ixodes ricinus ticks in seven study areas in Sweden. Parasit Vectors 3 (2010 Aug. 4) 66

### B. Other Articles

16. Baumann D et al., Fever after a tick bite; clinical manifestations and diagnosis of acute tick bite-associated infections in northeastern Switzerland. Deutsche Medizinische Wocnshrift (1946) 128 (19) (2003) 1042-1047.
17. Brouqui P et al., Guidelines for the diagnosis of tick-borne bacterial diseases in Europe. ESCNID Study group on Coxiella, Anaplasma, Rickettsia and Bartonella: European network for surveillance of tick-borne diseases, Clinical microbiology and infection: the official publication of the European Society of Clinical Microbiology and Infectious Diseases, France.
18. DiNubile M. J., Perspective relieving a nightmare: A hard (and tragic) lesson in humility. Clin. Infect. Disease 12 (1996) 160-64
19. Fournier P E et al., An eruptive fever associated with antibodies to Rickettsia helvetica in Europe and Thailand. J Clin Microbiol. 42(2) (2004 February) 816-8
20. Fryland et al., Low risk for developing Borrelia burgdorferi infection in the south-east of Sweden after being bitten by a Borrelia burgdorferi-infeted tick. Int. J. Infect. Dis. 15 (2011) e174-81.
21. Hughes C., Rocky Mountain Spotless fever with an Erythema Migrans-like skin lesion. Clin. Infect. Disease 21 (1998) 1328-29
22. Kondo et al., Japanese spotted fever with acute hepatic failure: was it associated with Epstein Barr virus. Int. J. of Dermatol. 49 (2010) 1403-06
23. Inst de Salud Carlos (inventors Fernandez A et al) Method and kit for the detection of bacterial species by means of DNA analysis. EPA 1895015, publ 05.03.2008
24. Lakos A., Tick-borne lymphadenopathy (TIBOLA). Wiener Klinische Wochenschrift 114(13-14) (2002) 648-654
25. Nielsen H. et al., Serological and molecular evidence of Rickettsia Helvetica in Denmark. Scand. J. Infect. Disease 36(8) (2004) 559-563
26. Parola P et al., Tick-borne bacterial diseases emerging in Europe. Clin Microbiol Infect. 7(2) (2001 February) 80-3. Review
27. Ramsey et al., Successful treatment of Rocky Mountain “Spotless” Fever. Western J. of Med. 140(1) (1984 January) 94-96
28. Bruce Fife, N. D., “Stop Alzheimer's now!”, Chapt. 10 (Infections) and Chapt. 11 (The dental connection) (2011) Picadilly Books, Ltd Colorado Springs, Colo., USA (ISBN 978-0-941599-85-6)

A Search report issued in the Swedish priority application has labelled references 2, 14, 16, 17, 23, 24, and 25 as particularly relevant (X) against the claims of the priority application. However, at least for refs 2 and 16 the results presented do not support that any of the tested individuals has an on-going Ricketsiosis and/or Borrelios when the tested samples were takent. Such an infection requires at least repeated tested of at least two samples taken at different times (3-10 weeks, same person) with a significant change in titer between the samples and with at least one sample having a titer of at least 1:128 with preference for at least 1:256.

1-12. (canceled)

13. A method for determining a possible bacterial infection in a patient which
a) exhibits symptoms selected from
A) specific internal symptoms of disease, and/or
B) diffuse and general manifestations of disease, and/or
b) is a blood donor
the method comprising:
i) determining
a) the level of a Rickettsia analyte in a biological sample 1 which derives from a patient exhibiting symptoms according to (A), possibly combined with
b) the level of a Borrelia analyte in a biological sample 2 which derives from the same patient,
ii) considering that the symptoms exhibited by said patient involve
a) rickettsiosis if the level of the Rickettsia analyte in sample 1 is elevated compared to corresponding samples from healthy individuals, and
b) borrelios if the level of the Borrelia analyte in sample 2 is elevated compared to corresponding samples from healthy individuals,
which for said blood donor means that whole blood and blood fractions obtained from said blood donor are considered to be infected with Rickettsia and Borrelia when these levels are elevated in samples 1 and 2, respectively.

14. The method of claim 13, wherein step (i.a) is imperatively combined with step (i.b).

15. The method of claim 13, wherein said symptoms of (A) comprises nerve inflammation (neuritis) causing symptoms such as loss of functions of nerves, for instance cranial nerves or pain from different parts of the body, meningitis, chronic perimyocarditis, hepatitis, autoimmune conditions and/or disease, neurodegenerative conditions and/or disease, psychotic conditions and/or disease etc.

16. The method of claim 13, further comprising carrying out steps (i)-(ii) before any antimicrobial treatment is initiated or if subsequent to the initiation of such a treatment as a follow up of the treatment in order to check for the appropriateness of the antimicrobial used or of the applied treatment.

17. The method of claim 13, wherein steps (i)-(ii) are carried out on two samples taken at two different occasions from the same patient, preferably with a time lapse of about 3-10 weeks between the samples/occasions.

18. The method of claim 13, wherein step (i.a) comprises determining Rickettsia analyte by an immunoassay for Rickettsia and that step (i.b), if included, comprises determining the Borrelia analyte by an immunoassay for Borrelia, each assay encompassing the substeps of
(i.A) formation of an affinity complex containing the analyte and an affinity counterpart to the analyte (anti-analyte) by reacting the analyte with a predetermined amount of the anti-analyte, and
(i.B) measuring during or subsequent to step (i.a) the presence of the complex formed,
(i.C) taking the formation of said complex as an indication of an increased level of Rickettsia analyte and Borrelia analyte, respectively, in the original sample(s).

19. The method of claim 13, wherein
i. the Rickettsia analyte is selected amongst i) Rickettsia nucleic acid, ii) Rickettsia antigen and/or ii) anti-Rickettsia antibody specific for the Rickettsia antigen, and
ii. if step (i.b) is included, the Borrelia analyte is selected amongst i) Borrelia antigen and ii) anti-Borrelia antibody specific for the Borrelia antigen,
where the preference is for antibody analytes (class (ii)).

20. The method of claim 13, wherein step (i.b) is included and the biospecific affinity assay for the Rickettsia analyte and for the Borrelia analyte, respectively, are the same with respect to at least one of:
A) the type of analyte (i)-(iii),
B) utilizing a non-competitive or a competitive biospecific affinity assay, such as a sandwich biospecific affinity assays,
C) utilizing an affinity reactant which is labeled to determine the formation of said affinity complex, with preference for the labeled affinity reactant being a labeled anti-analyte,
D) utilizing an affinity reactant which is immobilized or immobilizable to a solid phase, with preference for the immobilized or immobilizable reactant being an anti-analyte,
E) utilizing flow conditions for the formation the affinity complex with a liquid flow containing the analyte passing through a capturing zone containing the anti-analyte in immobilized form.

21. The method of claim 18, wherein the biospecific affinity assay for the Rickettsia analyte and for the Borrelia analyte are the same with respect to every feature (A)-(E), and/or preferably are carried out by forming the affinity complex containing the Rickettsia analyte and the affinity complex containing the Borrelia analyte in the same reaction mixture (=multiplexed assay), e.g. forming the two affinity complexes containing the Rickettsia analyte and the Borrelia analyte, respectively, essentially simultaneously or essentially consecutively in said reaction mixture.

22. A kit for the determination of bacterial infection of a patient a) having symptoms that potentially may be associated with infection by bacteria of the genus Rickettsia and/or the genus Borrelia, or b) being a blood donor, the kit comprising:
A) a Rickettsia reactant specific for detecting a Rickettsia analyte and selected amongst
i) an anti-Rickettsia antibody which is specific for a native Rickettsia antigen (analyte), and
ii) a Rickettsia antigen which is an affinity counterpart to a native anti-Rickettsia antibody (analyte)
B) a Borrelia reactant specific for detecting a Borrelia analyte and selected amongst
i) an anti-Borrelia antibody which is specific for a native Borrelia antigen (analyte), and
ii) a Borrelia antigen which is an affinity counterpart to a native n anti-Borrelia antibody (analyte).

23. The kit of claim 22, wherein the Rickettsia reactant and the Borrelia reactant are of the same class selected from (i)-(ii), with preference for both being an antigen, i.e. the Rickettsia reactant is a Rickettsia antigen for measuring native anti-Rickettsia antibody (analyte), and the Borrelia reactant is a Borrelia antigen for measuring anti-Borrelia antibody (analyte)

24. Method for the treatment of a patient exhibiting
A) specific internal symptoms of disease, and/or
B) visible skin manifestations which are associated with infection by a bacterium of the genus Rickettsia and/or the genus Borrelia, and/or
C) diffuse and general manifestations of disease, and/or
D) manifestations indicating that the patient has been attacked by a native vector that can be used for infection by a bacterium of the genus Rickettsia and/or the genus Borrelia the method comprising the steps of
a. determining if the patient suffers from an infection, preferably on-going, by Rickettsia and/or Borrelia by the use of the kit of claim 22, and subsequently
b. treating the individual with an anti-microbial agent effective against the bacteria determined in step (i).

25. Method for the treatment of a patient exhibiting
A) specific internal symptoms of disease, and/or
B) visible skin manifestations which are associated with infection by a bacterium of the genus Rickettsia and/or the genus Borrelia, and/or
C) diffuse and general manifestations of disease, and/or
D) manifestations indicating that the patient has been attacked by a native vector that can be used for infection by a bacterium of the genus Rickettsia and/or the genus Borrelia the method comprising the steps of
a. determining if the patient suffers from an infection, preferably on-going, by Rickettsia and/or Borrelia by the use of the kit of claim 23, and subsequently
b. treating the individual with an anti-microbial agent effective against the bacteria determined in step (i).
